(19) United States
(12) Patent Application Publication
(10) Pub. No.: US 2026/0053151 A1
(43) Pub. Date: Feb. 26, 2026

(54) APPARATUS AND METHOD FOR STUNNING FISH

(71) Applicant: Nordischer Maschinenbau Rud. Baader GmbH + Co. KG, Lübeck (DE)

(72) Inventors: Roman Gottschalk, Lübeck (DE); Ulf Jacobsen, Lübeck (DE); Jonas Emde, Lübeck (DE); Alexander Oldenburger, Lübeck (DE); Markus Heindel, Lübeck (DE); André Von Selle, Lübeck (DE)

(21) Appl. No.: 18/874,165

(22) PCT Filed: Jun. 15, 2022
(86) PCT No.: PCT/EP2022/066309

§ 371 Date: Oct. 23, 2025

(51) Int. Cl. A22B 3/08; G01N 33/12

(52) U.S. Cl. CPC A22B 3/083; G01N 33/12

(57) ABSTRACT

The present invention relates to a fish stunning apparatus, comprising a fish supply device adapted for conveying fish head first, a fish receiving space (10) configured by means of the fish supply device, to be loaded with fish in a head first position, a positioning means (11) with a positioning means (12) engaging the fish head, which is adapted to hold the fish in a stop position in the fish receiving space (10) for stunning the fish and to release the fish after stunning, a stunning means (13) acting on the head-side region of the fish for stunning the fish in the stop position, a trigger device (16) adapted for determining the stop position and for triggering the effect of the stunning means (13) on the head-side region of the fish, a control unit adapted for controlling the positioning device (11), the trigger device (16) and the stunning means (13), a fish size sensing device adapted for determining the fish size, a controllably moveable position actuator, which is adapted for locally adjusting the fish positioning means (12) in such a way that the respective position of the fish head relative to the stunning means (13) in the stop position is adapted to be adjusted in a self-actuating manner depending on the fish size and is characterised in that the fish size sensing device comprises a fish length sensing device adapted for determining the fish length and the fish size sensing device is configured for determining the fish size on the basis of the fish length. The invention further relates to a method for automatedly stunning fish.

The present invention relates to a fish stunning apparatus, comprising a fish supply device adapted for conveying fish head first, a fish receiving space configured by means of the fish supply device, to be loaded with fish in a head first position, a positioning device with a positioning means engaging the fish head, which is adapted to hold the fish in a stop position in the fish receiving space for stunning the fish and to release the fish after stunning, a stunning means acting on the head-side region of the fish for stunning the fish in the stop position, a trigger device adapted for determining the stop position and for triggering the effect of the stunning means on the head-side region of the fish, a control unit adapted for controlling the fish positioning device, the trigger device and the stunning means, a fish size sensing device adapted for determining the fish size, a controllably moveable position actuator, which is adapted for locally adjusting the fish positioning means in such a way that the respective position of the fish head relative to the stunning means in the stop position is adapted to be adjusted in a self-actuating manner depending on the fish size.

The invention further relates to a method for automatedly stunning fish, comprising the conveying of fish head first by means of a fish supply device, loading a fish receiving space by means of the fish supply device with fish in a head first position, determining a stop position and a time for triggering the effect of a stunning means on the head side region of the fish by means of a trigger device, holding the fish in the stop position in the fish receiving space by means of a positioning device with a positioning means engaging the fish head, controlling the positioning device, the trigger device and the stunning means by means of a control unit, determining the fish size by means of a fish size sensing device, adjusting the fish positioning means by means of a controllably moveable position actuator, which is adapted for locally adjusting the positioning means in such a way that the respective position of the fish head relative to the stunning means in the stop position is adapted to be independently adjustable depending on the fish size, stunning the fish in the stop position through a stunning means acting on the head side region of the fish and releasing the fish following stunning by means of the positioning means.

A fish stunning apparatus with the above-mentioned features is known from document WO 2016/026540 A1. Fish are individually supplied to the fish stunning apparatus one after the other. The fish arrive individually with or in a water stream in prone position, head first, in the fish receiving space. The trigger device is located there. The fish head hits the same thanks to its own weight and thus reaches a temporary stop position. The trigger device has an abutment element, which is mounted in a sliding moveable or pivot moveable way. A sensor means reacting to the abutment of the fish head against the abutment element of the trigger device triggers the operation of the stunning means. The stunning means comprises a stunning tool as well as an optional bleeding tool.

In order to stun the fish in an optimal and animal protection compliant way the fish head is fixed in the stop position by means of the positioning means and the optimal position for this is set by means of the controllably moveable position actuator depending on the fish size. Such an apparatus that is technically improved compared to that of document WO 2016/026540 A1 as well as a corresponding method is already known from patent application EP 16736876 (publication number 3484299) originating from the house of the applicant. We therefore refer to application EP 16736876 in its entirety and its content is herewith included in this application.

It is a disadvantage that additional apparatus and sensor effort is required for determining the fish size, which complicates the apparatus as well as the method and generally makes the same prone to faults and high maintenance during rough industrial use.

It is therefore the problem of the present invention to suggest a corresponding apparatus that will allow a reliable fish size determination for an optimal adjustment of the said positioning device with the lowest possible apparatus effort. The problem further consists of suggesting a corresponding method.

The problem is solved by an apparatus with the features mentioned above in that the fish size sensing device comprises a fish length sensing device adapted for determining the fish length and the fish size sensing device is configured for determining the fish size on the basis of the fish length. It has been found that the fish length is a reliable starting parameter, based upon which conclusions can be drawn regarding the actual fish size. The apparatus effort for determining the fish size can be reduced to a minimum in this way, as substantially no complex devices for fish size determination, such as for example weighing devices or suchlike, are required. Rather, one can fall back on numerous components already provided in such a fish stunning apparatus for determining the fish length as a starting basis for determining the fish size. A reliable and precise determination of the fish size is therefore still possible with the lowest possible apparatus effort in order to carry out the said exact adjustment of the positioning device depending on the fish size.

Fish size is understood as a parameter based on which the fish can be allocated to corresponding fish size ranges and/or fish size classes. The fish size is for example characterised by body volume, mass, the maximum width between the side flanks, anatomically based size relationships such as for example the relationship between the head and the overall fish length or suchlike. The fish size is consequently a value or value range which also allows the allocation of the fish to different stipulated fish size classes.

The controllably moveable position actuator is configured in such a way that the same acts on the fish positioning means in order to adjust the position of the fish head relative to the stunning means in the stop position depending on the fish size in a self-actuating manner. The position actuator is further preferably configured to also adjust the position of the trigger device together with the fish positioning means. The position of the trigger device can also be adjusted depending on fish size in this way.

A fish stunning apparatus in the sense of this invention is understood as stunning the fish with the consequence of death. Stunning takes place in two steps. The fish is first stunned by means of a stunning tool and is rendered unconscious in this way. Bleeding out with the consequence of death is then induced. The fish stunning apparatus in the sense of the present invention therefore in other words also forms a fish slaughtering apparatus. The fish stunning device is preferably configured in such a way that the operation of the bleeding tool is control variably switched on and off. In this way it is possible to check whether the stunning tool works correctly and stuns the fish, preferably irreversibly.

An expedient design embodiment of the invention is characterised in that the fish size sensing device is adapted to determine on the basis of the fish length whether the specific size of the fish falls into one of at least two stipulated fish size ranges and to determine the respective fish size range as the fish size. In this way it is possible to determine which fish size the respective fish has and to determine into which of the stipulated fish size ranges the fish falls. This has the advantage that the positioning device is optimally positioned depending on the fish size on the one hand, and that the position of the positioning device can be maintained for the continuous arrival of fish, the fish size of which falls into the same fish size range, on the other hand.

The number of repositionings of the positioning device can thus be reduced to a minimum. The present invention is, however, not limited to a classification into said two stipulated fish size ranges. Depending on the desired precision of the position adjustment of the positioning device any number of fish size ranges can be stipulated.

If desired it is even possible to adjust and adapt the positioning device individually for every fish size in order to guarantee an optimal positioning of the fish in the stop position.

A preferred further development of the invention is characterised in that the fish size sensing device has a sensor device, which is adapted for determining the fish length of the fish. Such a sensor device for determining the fish length offers the advantage of the lowest possible apparatus effort. The fish length can be determined sufficiently accurately with such a sensor device and offers a reliable starting basis for determining the fish size.

A further expedient embodiment of the invention is characterised in that the sensor device comprises at least two sensors arranged at a distance from each other in longitudinal direction of the fish receiving space, which are adapted for detecting the presence of the fish in the respective sensing region of the sensor and provide detection signals. The sensors are thus arranged in the region of the fish receiving space, for example in the side walls. Alternatively, the sensors are arranged before the fish receiving space, e.g., upstream thereof, so that the fish pass the same before they reach the fish receiving space. The position of the sensors is preferably selected in such a way that, in any event, enough time remains for determining the fish size based on the detection signals and, based on said fish size, to position the positioning means accordingly before the fish reaches the stop position.

According to a further preferred embodiment of the invention the sensor device comprises an evaluation unit, which is adapted for determining the fish length on the basis of the detection signals. To put it differently, the evaluation unit is configured to determine the actual fish length based on the detection signals of the sensor device in order to use the same as a starting basis for the determination.

A further expedient embodiment of the invention is characterised in that the evaluation unit is further adapted to check on the basis of the detection signals whether when the presence of the fish in the sensing region is detected by means of a first sensor of the sensors, arranged on the stunning means side, the detection of the presence of the fish in the sensing region of a second sensor of the sensors, arranged on the fish supply device side is also detected.

Moreover, the evaluation unit is adapted to determine as fish size the respective larger stipulated fish size range if the presence of the fish is detected in both sensing regions of the respective first and second sensors, and otherwise determine the respective smaller of the stipulated fish size ranges. To put it differently, the evaluation unit recognises when the first sensor detects the presence of a fish in its sensing region and then checks whether the second sensor has also detected the presence of a fish in its sensing region. If this is the case it will be a fish with a fish size within the larger stipulated fish size range, otherwise such a one whose size lies within the smaller stipulated fish size range. Such a measurement can advantageously take place whilst the fish is standing still, i.e., statically, as well as with moving fish, i.e., dynamically.

A further expedient embodiment of the invention is characterised in that further sensors, distanced from each other, are arranged in longitudinal direction of the fish receiving space, which are adapted for detecting the presence of the fish in the respective sensing region of the sensor and provide detection signals, and that the evaluation device is further adapted to determine, on the basis of the detection signals, whether the fish size falls into a corresponding fish size range by a distance between the first sensor and one of the further sensors, and to determine the respective fish size range as the fish size. This way, it is advantageously possible to determine into which size category the respective fish fall. The adjustment of the position of the positioning means then preferably takes place on the basis of the determined fish size ranges.

According to a further preferred embodiment the fish receiving space has a floor element, wherein the floor element forms an inclined plane tilted towards the stunning means in the direction of gravity, so that the fish are guided slidably on the floor element after loading the fish receiving space under the effect of gravity. This has the advantage that the fish automatically slide or slip in the direction of the stunning means. Stipulating the angle of inclination of the inclined plane makes it possible to influence the course of motion, and therefore the speed or acceleration of the fish.

Preferably, the angle of inclination of the floor element is configured adjustably.

A further expedient embodiment of the invention is characterised in that the fish size sensing device comprises a time difference sensing unit, which is adapted to determine at least one time interval on the basis of the detection signals of at least two of the sensors, which the fish needs to move from one sensing region of one of the sensors to at least one other sensing region of another sensor, and that the fish size sensing device has a processing unit, which is adapted for determining the fish length of the fish, based on the determined at least one time interval on the basis of a cinematic model describing the motion of the fish in the fish receiving space. To put it differently, the time difference sensing unit is adapted to determine the “falling time” of the fish on the inclined plane, i.e., the time that is required by the fish to pass both sensors.

Modelling the course of motion of the fish on this inclined plane makes it possible to determine the fish length, using the said cinematic model, which fish length then serves as a starting basis for the fish size determination.

According to a further preferred embodiment of the invention the cinematic model comprises at least one path/time equation, which at least approximately describes the motion of the fish depending on an angle of inclination of the inclined plane. The cinematic model is for example of a linear configuration. Simplified, in this case a uniform or a uniformly accelerated motion of the fish is assumed.

A preferred further development of the invention is characterised in that the cinematic model comprises empirically determined motion data. A cinematic model based on empirically determined motion data has the advantage that the motion data were determined on a plurality of different fish and reflect the real relations of the cinematic. It is thus possible to determine the fish length, and therefore the fish size, for a plurality of fish types and fish sizes correspondingly precisely.

A further expedient embodiment of the invention is characterised in that the cinematic model is established on the basis of a self-learning neuronal network. The use of such a neuronal network has the advantage of a very precise determination of the actual fish size based on the previously taught empirical values.

A further expedient embodiment of the invention is characterised in that, after loading the fish receiving space with one of the fish, the control unit is configured to first prompt the fish size sensing device to determine the fish size of the fish, and to subsequently set the position of the fish head relative to the stunning means in the stop position according to the fish size by means of the controllably moveable position actuator in a self-actuating manner. An always optimal fixing of the respective fish in the stop position is thus realised, so that a reliable stunning and subsequent bleeding out is guaranteed. The determination of the stop position preferably takes place before the fish comes to temporarily lie in the same.

A further expedient embodiment of the invention is characterised in that the fish length sensing device is further configured to determine a head-to-overall-length relationship based on the fish size and that the control unit is adapted for setting the position of the fish head in the stop position according to the head-to-overall-length relationship. The relationship of the length of the fish head to the overall fish length constitutes a reliable starting basis for determining the position of the fish head in the stop position. The fish length sensing device for example comprises a database, in which the head-to-overall-length relationships for different fish types are stored. The fish length sensing device is thus configured to determine the head-to-overall-length relationship based on the fish size. The fish length sensing device is further adapted to determine the position of the fish head in the stop position based on the determined head-to-overall-length relationship. A database can for example be used for this purpose as well, in which the positions associated with the head-to-overall-length relationships for the respective optimal positioning of the fish head are stored.

A further expedient embodiment of the invention is characterised in that the control unit comprises a prone/supine position sensing unit, which is configured to recognise whether the fish is aligned with its abdominal side upwards, and if the fish is aligned with its abdominal side upwards, to separate the fish out of the fish receiving space. A correct position of the fish in the fish receiving space is always ensured in this way, namely in that the same is aligned with its abdomen downwards. Fish that reach the fish receiving space with their back directed downwards are separated out and may be supplied to the fish receiving space once again or post-processed following a change to the prone/supine position. The fish for example reach a catchment basin via a separate path, from which they can later be supplied once more. In this way it is ensured that an animal protection compliant stunning and bleeding out is always guaranteed.

A further expedient embodiment of the invention is characterised in that the control unit further comprises a kype recognition device, which is adapted for detecting sexual dimorphism-related changes in the fish and to adjust the determined head-length-to-overall-length relationship according to the determined kype condition of the fish based on an anatomic model of the fish. For this purpose, the kype recognition device comprises corresponding sensors, which are configured for sensing and evaluating both, anatomical changes caused by the kype condition in the fish and/or colour changes. Fish with kype, for example, have a longer head in relation to their overall fish length.

A further expedient design of the invention is characterised in that the kype recognition device comprises at least one optical sensor, which is adapted for optically scanning the abdominal side of the fish and detecting the kype condition through comparison with a stipulated brightness value or brightness profile. The abdominal side of fish with kype is normally darker than those of fish without kype. By stipulation of corresponding brightness values, brightness profiles, brightness areas and/or brightness threshold values the described comparison can allow for recognition whether the fish is in a kype condition or not, and this can be taken into consideration when determining the head-length-to-overall-length relationship.

An advantageous embodiment of the invention is characterised in that the fish length sensing device is further configured to determine fish size ranges, based on a predetermined distribution of fish sizes and/or on a determined fish size range, and if the respective fish size determined is within one of these fish size ranges, setting the position of the fish head in the stop position according to a position stipulation stipulated for the respective fish size range. This increases the robustness of the classification of the fish into said fish size ranges against measuring errors and/or measuring inaccuracies when determining the fish size. Preferably, each time, the position of the fish head is adapted to be set in a self-actuating manner relative to the stunning means in the stop position depending on fish size, and is thus set depending on the respective fish size range detected.

The problem is further solved by a corresponding method with the features mentioned above through determining the fish length by means of a fish length sensing device and determining the fish size on the basis of the fish length by means of the fish size sensing device. The advantages connected therewith have already been sufficiently explained above in connection with the apparatus according to the invention. Similarly, this applies to the following method features, so that we refer to the advantages of the respective features of the apparatus according to the invention in connection with the same.

A further expedient embodiment of the invention is characterised by checking on the basis of the fish length whether the specific size of the fish falls into one of at least two stipulated fish size ranges, and determining the fish size as the respective fish size range.

A preferred further development of the invention is characterised by detecting the presence of the fish in the respective sensing region of the sensor and the provision of detection signals.

A further expedient embodiment of the invention is characterised by detecting the presence of the fish in the respective sensing region of the sensor and the provision of detection signals by means of at least two sensors of the sensor device arranged at a distance from each other in longitudinal direction of the fish receiving space.

A further expedient embodiment of the invention is characterised by determining the fish length on the basis of the detection signals by means of an evaluation device of the sensor device.

A preferred further development of the invention is characterised by checking on the basis of the detection signals by means of the evaluation unit whether when detecting the presence of the fish in the sensing region of a first sensor of the sensors arranged on the stunning means side presence of the fish in the sensing region of a second sensor of the sensors arranged on the fish supply device side is detected as well, and in case presence of the fish in both sensing regions of the first and second sensors is detected, by determining as fish size the respective larger stipulated fish size range, and by otherwise determining the respective smaller of the stipulated fish size ranges. A further expedient embodiment of the invention is characterised by detecting the presence of the fish in the respective sensing region of the sensor and providing detection signals by means of further sensors arranged in longitudinal direction of the fish receiving space at a distance from each other, and, by means of the evaluation device, determining the respective fish size range as the fish size on the basis of the detection signals, if the fish size falls into a fish size range corresponding to the distance between the first sensor and one of the further sensors.

A further expedient embodiment of the invention is characterised in that the fish are guided to slide onto a floor element of the receiving element following loading of the fish receiving space under the effect of gravity, wherein the floor element forms a suitable inclined plane in relation to the stunning means in the direction of gravity.

A preferred further development of the invention is characterised by determining at least one time interval, which is needed by the fish to move from one sensing region of one of the sensors to at least one other sensing region of at least one other of the sensors, by means of a time difference sensing unit of the fish size sensing device on the basis of the detection signals of at least two of the sensors, and determining the fish length of the fish on the basis of a cinematic model describing the motion of the fish in the fish receiving space by means of a processing unit of the fish size sensing device.

A preferred further development of the invention is characterised in that the cinematic model comprises at least one path/time equation, which at least approximately describes the motion of the fish depending on an angle of inclination of the inclined plane.

According to a further preferred embodiment the cinematic model comprises empirically determined motion data.

A further expedient embodiment of the invention is characterised in that the cinematic model is generated on the basis of a self-learning neuronal network.

A preferred further development of the invention is characterised by initiating the fish size sensing device to firstly determine the fish size of the fish following the loading of the fish receiving space, and to subsequently set the position of the fish head relative to the stunning means in the stop position depending on the fish size by means of the controllably moveable position actuator in a self-actuating manner.

A further expedient embodiment of the invention is characterised by determining a head-to-overall-length relationship based on the fish size by means of the fish length sensing device and setting the position of the fish head in the stop position depending on the head-to-overall-length relationship by means of the control unit.

A further expedient embodiment of the invention is characterised by recognising by means of a prone/supine position sensing unit of the control unit whether the fish lies aligned with its abdominal side downwards and, if the fish lies with its abdominal side upwards, separating out of the fish from the fish receiving space.

A preferred further development of the invention is characterised by detecting sexual dimorphism-related changes in the fish by means of a kype recognition device of the control unit and adjusting the determined head-to-overall-length relationship based on an anatomic model of the fish according to the determined kype condition of the fish.

A preferred further development of the invention is characterised by an optical scanning of the abdominal side of the fish by means of an optical sensor of the kype recognition device and determining a brightness value or a brightness profile as well as comparing the same with a stipulated brightness value or brightness profile in order to detect the kype condition.

Further preferred and/or expedient features and embodiments designs of the invention arise from the subclaims and the description. Particularly preferred embodiments are explained in more detail with reference to the enclosed drawing. In the drawing:

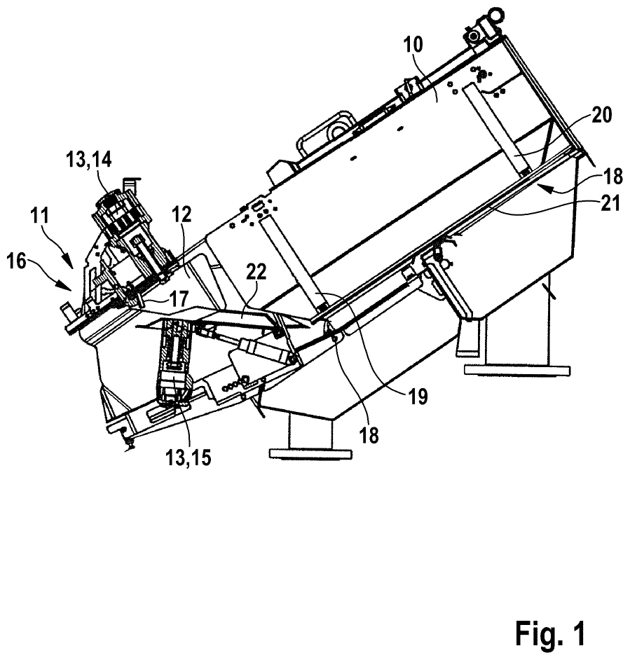

FIG. 1 shows an embodiment example of a fish stunning apparatus.

A fish stunning apparatus is schematically illustrated in FIG. 1. For reasons of improved clarity neither the fish to be stunned nor a fish supply device, which is adapted for supplying each fish in head first position to a fish receiving space 10 configured for individual reception of each fish, are shown in FIG. 1. In other words, the fish receiving space 10 is configured in such a way that the same can be loaded with fish in the head first position. The fish are therefore supplied to the fish receiving space 10 head first. The apparatus according to the invention as well as the method according to the invention will be described in more detail in the following with reference to the drawing.

The fish stunning apparatus according to the invention further comprises a positioning device 11 with a positioning means 12 engaging the fish head. The positioning means 12 is adapted for holding the fish in a stop position in the fish receiving space 10 in order to fix the same for stunning. The positioning means 12 is further adapted for loosening the fish from this fixing after stunning and releasing the same.

The invention according to the fish stunning apparatus further comprises a stunning means 13 for stunning the fish in the stop position. The stunning means 13 preferably comprises a stunning tool 14, which acts mechanically on the head area of the fish for stunning the same, as well as a bleeding out tool 15, which acts on the region between head and gills on the underside of the fish. The stunning means 13 is preferably adapted to irreversibly stun the fish.

The present invention further comprises a trigger device 16, which is adapted for determining the stop position and for triggering the action of the stunning means 13 on the head side region of the fish. The trigger device 16 comprises at least one moveable, preferably pivot moveable, mounted abutment element 17, against which the fish comes to lie with its head side when reaching the stop position, and which then triggers the action of the stunning means 13.

A control unit—not shown in the drawing—such as for example a computer controller, the function of which is additionally explained below, is provided for controlling the positioning device 11, the trigger device 16 and the stunning means 13. The trigger device 16 preferably comprises at least one sensor element, by means of which a position change of the abutment element 17 can be sensed when a fish head lies against the abutment element 17, which position change can be evaluated by means of the control unit.

The fish stunning device according to the invention further comprises a fish size sensing device configured for determining the fish size as well as controllably moveable position actuator—not shown in the drawing. The position actuator is adapted for the location adjustment of the positioning means 12 in such a way that each time the position of the fish head relative to the stunning means 13 is adapted to be adjustable in a self-actuated manner in the stop position according to the fish size.

The mode of operation of such a position actuator is for example sufficiently known from document EP 3484299 A1, originating from the house of the applicant. We refer to application EP 16736876 in this regard, the content of which is herewith included in this application.

The fish size sensing device is adapted and configured for determining the fish size on the basis of the fish length. It is thus possible in a particularly easy way to determine the fish size as exactly as possible or to allocate the respective fish to a fish size class in order to optimally adjust the positioning means 12 to the respective fish size, respectively.

The fish size sensing device is preferably adapted for determining on the basis of the fish length whether the determined size of the fish falls into one of at least two stipulated fish size ranges, and for determining the respective fish size range as the fish size. The classification into two fish size ranges has the advantage that, on the one hand, a sufficiently accurate adjustment of the positioning means 12 is possible in order to ensure an optimal effect of the stunning means 13, whilst the number of position adjustments of the positioning means 12 required is reduced to a minimum on the other hand. If, for example, two fish that can be allocated to the same fish size range follow each other, the positioning means 12 can remain in the previously set position. Depending on the desired positioning accuracy it is possible to stipulate more than two fish size ranges and to displace the positioning means 12 into different positions according to the number of fish size ranges.

The fish sensing device preferably has a sensor device 18, which is adapted for determining the fish length of the fish. In principle, any device by means of which the determined fish length can be allocated to a length category that is connected with the previously mentioned stipulated fish size ranges is suitable as a sensor device 18.

As shown in FIG. 1 the sensor device 18 comprises at least two first and second sensors 19, 20 arranged at a distance from each other in longitudinal direction of the fish receiving space. The sensors 19, 20 are adapted for detecting the presence of the fish in the respective sensing region of the sensor 19, 20 and for providing corresponding detection signals. The sensors 19, 20 can for example be configured as contactless sensors or as electro-mechanical sensors. The sensors 19, 20 are particularly preferably each configured as light barriers, in particular reflection or forked light barriers, which detect the presence of a fish when the same appears in their detection area. Other sensor types, for example proximity sensors or radar-based sensors, by means of which the presence of a fish can be detected, are, however, also suitable in principle.

The sensor device preferably comprises an evaluation unit, which is adapted for determining the fish length on the basis of the detection signals. The evaluation unit is thus in particular configured for deducing the fish length on the basis of the distance between the first and second sensor as well as the detection signals provided.

The evaluation unit is advantageously further adapted for checking, on the basis of the detection signals, whether, whilst detecting the presence of the fish in the detection area determined by means of the first sensor 19 of the sensors arranged on the fish supply device side, the presence of the fish in the detection area of the second sensor 20 arranged on the stunning means side is also determined. If the presence of the fish is detected in both detection areas of the first and second sensors 19, 20, the respective larger stipulated fish size range is determined as the fish size, otherwise, the respective smaller one of the stipulated fish size ranges is determined. The measuring principle is in other words based on classifying the actual fish size into at least two fish size ranges and to safely determine into which of the two fish size ranges the respective fish falls.

The number of sensors 19, 20 is further preferably not limited to two. A further advantageous embodiment of the invention—not shown in the drawing—is characterised in that further sensors are arranged at a distance from each other in longitudinal direction of the fish receiving space 10. These are adapted similarly to the sensors 19, 20 in order to detect the presence of the fish in the respective detection area and provide corresponding detection signals. The evaluation device is correspondingly further adapted for determining on the basis of the detection signals whether the fish size falls into one fish size range corresponding to the distance between the first sensor 19 and one of the further sensors, and for determining the respective fish size range as the fish size. It is possible in this way to classify the actual fish size into further, namely into a number of n-1 ranges, wherein n equals the number of sensors arranged along the fish receiving space 10.

The fish receiving space 10 further preferably has a floor element 21. The floor element 21 forms an inclined plane tilted towards the stunning means 13 in the direction of gravity. This results in the fish to be processed being guided sliding on the floor element 21 after loading the fish receiving space 10 under the effect of gravity. In other words, the fish “slides” in a self-actuated manner along the tilted plane in the direction of the stunning means 13 under the effect of gravity in the fish receiving space 10 and passes the previously mentioned sensor device 18.

The floor element 21 is followed by a ramp 22, on which the fish comes to lie with its head side. The ramp 22 is preferably configured pivotably movable and is pivoted from the fish receiving space 10 in the direction of gravity to release the fish. The ramp 22 is preferably arranged to tilt upwards above the floor element 21 against the direction of gravity and is adapted for slowing down the motion of the fish until it stands still.

The fish size sensing device preferably comprises a time difference sensing unit—not shown in the drawing. This is adapted for determining at least one time interval on the basis of the detection signals from at least two of the sensors 19, 20, which time interval the fish needs to move from the sensing region of one of the sensors 19, 20 to at least one other sensing region of at least another one of the sensors 20, 19. To put it differently, the time difference sensing unit is adapted to sense what time the fish requires to travel the distance between the second sensor 20 and the first sensor 19.

The fish size sensing device further has a processing unit, which is adapted for determining the fish length of the fish based on the determined at least one time interval on the basis of a cinematic model describing the motion of the fish in the fish receiving space. In the simplest case such a cinematic model describes the motion of the fish under the effect of gravity as a uniform motion with constant speed. The cinematic model can also reflect the motion of the fish as a uniformly accelerated motion. Considering at least the determined time interval the fish length is then deduced by means of the cinematic model and the same is determined. In other words, the cinematic model is adapted for estimating the fish's motion dynamics to be expected. If, for example, the speed of the fish depending on the time is reflected by the cinematic model, the determination of the said time interval allows the actual fish size to be deduced.

The cinematic model further preferably comprises at least one path/time equation, which describes the motion of the fish depending on an angle of inclination of the inclined plane at least approximately. In addition to the factors already mentioned the angle of inclination is also taken into consideration when modelling the fish motion in order to thus determine the fish length.

Alternatively, the cinematic model comprises empirically determined motion data, which were for example determined during preceding trials. The correlation between the determined time interval and the fish length is for example stored in a database and then accessed for determining the fish length.

The cinematic model is further preferably based on a self-learning neuronal network.

Such a neuronal network is first taught accordingly to determine the fish length based on the other parameters. The neuronal network is preferably adapted in such a way that the same is configured to keep self-learning during further operation.

The control unit is preferably configured to first prompt the fish size sensing device to determine the fish size of the fish after loading the fish receiving space 10 with one of the fish. By means of the controllably moveable position actuator, the position of the fish head relative to the stunning means in the stop position is then set according to the fish size in a self-actuated manner by means of the control unit.

The fish length sensing device is preferably further configured for determining a head-to-overall-length relationship based on the fish size. It has been found that the relationship between the length of the head of a fish and the overall length forms a reliable starting basis, by means of which the optimal position of the fish head in the stop position can be determined in order to be able to optimally stun the fish. The control unit is in particular adapted for setting the position of the fish head in the stop position depending on the head-to-overall-length relationship.

The control unit optionally comprises a prone/supine position sensing unit—not shown in the drawing. This is configured for detecting whether the fish is aligned with its abdominal side upwards, and if the fish is aligned with its abdominal side upwards, for separating the fish out of the fish receiving space 10 prior to stunning. In this way it is ensured that the fish is always guided to the stunning means 13 in the correct position. The present invention further preferably comprises a kype recognition device, which is adapted for detecting sexual dimorphism-related changes in the fish and to adjust the determined head-length-to-overall-length relationship based on an anatomic model of the fish according to the detected kype condition of the fish.

The kype recognition device comprises at least one optical sensor, which is adapted for optically scanning the abdominal side of the fish and for recognising the kype condition through comparison with a stipulated brightness value, brightness profile or brightness range.

The above embodiments in connection with the apparatus according to the invention also apply in an analogue fashion for the method according to the invention, so that only selected aspects of the method need to be discussed once more in the following.

Apart from this we refer to the above explanations in connection with the method according to the invention.

The fish are conveyed by means of a fish supply device—not shown in the drawing—head first and the fish receiving space 10 is loaded with one of the fish each. The stop position and the time for triggering the action of a stunning means 13 on the head side region of the fish are then determined by means of a trigger device 16.

The fish is held in this stop position in the fish receiving space 10 by means of the positioning device 11. The positioning means 12 engage the fish head for this. The positioning means 11, the trigger device 16 and the stunning means 13 are controlled by means of the control unit for this.

Determining the fish size takes place by means of the fish size sensing device in order to adjust the positioning means 12 on the basis of the fish size. The positioning means 12 is then adjusted by means of a controllably moveable position actuator. The position actuator is adapted for locally adjusting the positioning means 12 in such a way that the position of each fish head relative to the stunning means 13 is adapted to be adjustable in a self-actuating manner in the stop position depending on the fish size.

The fish is stunned in the stop position by the stunning means 13 acting on the head side region of the fish. As described above, the skull of the fish is preferably first hit by means of the stunning tool 14 in order to render the same unconscious before a bleeding out of the fish is initiated with a bleeding out tool 15.

After being stunned the fish is then released by means of the positioning device 11, i.e. released from its fixing. Detecting the fish length by means of the fish length sensing device then takes place for determining the fish size and the fish size is determined based on the fish length by means of the fish size sensing device.

Fish size ranges are preferably also determined by means of the fish length sensing device, based on a stipulated distribution of the fish sizes and/or on a determined fish size distribution. If the respective fish size determined lies within one of these fish size ranges, the position of the fish head in the stop position is set according to a position stipulated for the respective fish size range.

1. Fish stunning apparatus, comprising
a fish supply device for conveying fish head first,
a fish receiving space (10) to be loaded with fish in a head first position,
a positioning device (11) with a positioning means (12) engaging the fish head, which is adapted to hold the fish in a stop position in the fish receiving space (10) for stunning the fish and to release the fish after stunning,
a stunning means (13) acting on the head-side region of the fish for stunning the fish in the stop position,
a trigger device (16) adapted for determining the stop position and for triggering the effect of the stunning means (13) on the head-side region of the fish,
a control unit adapted for controlling the positioning device (11), the trigger device (16) and the stunning means (13),
a fish size sensing device adapted for determining the fish size,
a controllably moveable position actuator, which is adapted for locally adjusting the fish positioning means (12) in such a way that the respective position of the fish head relative to the stunning means (13) in the stop position is adapted to be independently adjusted depending on the fish size,
characterised in that
the fish size sensing device comprises a fish length sensing device for determining the fish length, and the fish size sensing device is configured for determining the fish size on the basis of the fish length.

2. Apparatus according to claim 1, characterised in that the fish size sensing device is adapted for determining, on the basis of the fish length, whether the specific size of the fish falls into one of at least two stipulated fish size ranges and for determining the respective fish size range as the fish size.

3. Apparatus according to claim 2, characterised in that the fish size sensing device has a sensor device (18), which is adapted for determining the fish length of the fish.

4. Apparatus according to claim 3, characterised in that the sensor device (18) comprises at least two sensors (19, 20) arranged at a distance from each other in longitudinal direction of the fish receiving space (10), which are adapted for detecting the presence of the fish in the respective sensing region of the sensor and for providing detection signals.

5. Apparatus according to claim 4, characterised in that the sensor device (18) comprises an evaluation unit, which is adapted for determining the fish length on the basis of the detection signals.

6. Apparatus according to claim 5, characterised in that the evaluation unit is further adapted for checking on the basis of the detection signals whether the presence of the fish in the detection area determined by means of the first sensor (19) of the sensors arranged on the fish supply device side is also determined whilst detecting the presence of the fish in the detection area of the second sensor (20) arranged on the stunning means side, and if the presence of the fish is detected in both detection areas of the first and second sensors (19, 20) the respective larger stipulated fish size range is determined as the fish size, and the respective smaller one of the stipulated fish size ranges is otherwise determined.

7. Apparatus according to claim 6, characterised in that further sensors are arranged at a distance from each other in longitudinal direction of the fish receiving space (10), which are adapted for detecting the presence of the fish in the respective sensing region of the sensor and for providing detection signals, and in that the evaluation device is further adapted for determining on the basis of the detection signals whether the fish size falls into a corresponding fish size range determined by the distance between the first sensor (19) and one of the further sensors, and for determining the respective fish size range as the fish size.

8. Apparatus according to one of the claims 1 to 7, characterised in that the fish receiving space (10) has a floor element (21), wherein the floor element (21) forms an inclined plane tilted towards the stunning means (13) in the direction of gravity, so that the fish are guided slidably on the floor element (21) after loading the fish receiving space (10) under the effect of gravity.

9. Apparatus according to claim 8, characterised in that the fish size sensing device comprises a time difference sensing unit, which is adapted to determine at least one time interval on the basis of the detection signals of at least two of the sensors, which the fish needs to move from one sensing region of one of the sensors to at least one other sensing region of another sensor, and the fish size sensing device has a processing unit, which is adapted for determining the fish length of the fish, based on the determined at least one time interval on the basis of a cinematic model describing the motion of the fish in the fish receiving space (10).

10. Apparatus according to claim 9, characterised in that the cinematic model comprises at least one path/time equation, which at least approximately describes the motion of the fish depending on an angle of inclination of the inclined plane.

11. Apparatus according to one of the claims 9 or 10, characterised in that the cinematic model comprises empirically determined motion data.

12. Apparatus according to one of the claims 9 or 11, characterised in that the cinematic model is generated on the basis of a self-learning neuronal network.

13. Apparatus according to one of the claims 1 to 12, characterised in that the control unit is configured to first prompt the fish size sensing device to determine the fish size of the fish following loading the fish receiving space (10) with one of the fish, and subsequently set the position of the fish head in a self-actuating manner relative to the stunning means (13) in the stop position according to the fish size.

14. Apparatus according to one of the claims 1 to 13, characterised in that the fish length sensing device is further adapted for determining a head-to-overall-length-relationship based on the fish size, and that the control unit is adapted for setting the position of the fish head in the stop position depending on the head-to-overall-length relationship.

15. Apparatus according to one of the claims 1 to 14, characterised in that the control unit comprises a prone/supine position sensing unit, which is configured to determine whether the fish is aligned with its abdominal side upwards, and if the fish is aligned with its abdominal side upwards, separating the fish out of the fish receiving space.

16. Apparatus according to one of the claims 1 to 15, characterised in that the fish length sensing device is further configured to determine fish size ranges, based on a stipulated distribution of the fish sizes and/or on a determined fish size distribution, and if the respective determined fish size lies within one of these fish size ranges, for positioning the fish head in the stop position depending on a position stipulation stipulated for the respective fish size range.

17. Apparatus according to claim 16, characterised in that the control unit further comprises a kype recognition device, which is adapted for detecting sexual dimorphism-related changes in the fish and to adjust the determined head-length-to-overall-length relationship according to the determined kype condition of the fish based on an anatomic model of the fish.

18. Apparatus according to claim 17, characterised in that the kype recognition device comprises at least one optical sensor, which is adapted for optically scanning the abdominal side of the fish and detecting the kype condition through comparison with a stipulated brightness value or brightness profile.

19. Method for automatedly stunning fish, comprising
the conveying of fish head first by means of a fish supply device,
loading a fish receiving space (10) by means of the fish supply device with fish in a head first position,
determining a stop position and a time for triggering the effect of a stunning means (13) on the head side region of the fish by means of a trigger device (16),
holding the fish in the stop position in the fish receiving space (10) by means of a positioning device (11) with a positioning means (12) engaging the fish head,
controlling the positioning device (11), the trigger device (16) and the stunning means (13) by means of a control unit,
determining the fish size by means of a fish size sensing device,
adjusting the fish positioning means (12) by means of a controllably moveable position actuator, which is adapted for locally adjusting the positioning means (12) in such a way that the respective position of the fish head relative to the stunning means (13) in the stop position is adapted to be independently adjustable depending on the fish size,
stunning the fish in the stop position through a stunning means (13) acting on the head side region of the fish,
releasing the fish following stunning by means of the positioning means (11),
characterised by
determining the fish length by means of a fish length sensing device and determining the fish size on the basis of the fish length by means of the fish size sensing device.

20. Method according to claim 19, characterised by checking on the basis of the fish length whether the specific size of the fish falls into one of at least two stipulated fish size ranges and determining the fish size as the respective fish size range.

21. Method according to claim 20, characterised by detecting the presence of the fish in the respective sensing region of the sensor and providing detection signals.

22. Method according to claim 21, characterised by detecting the presence of the fish in the respective sensing region of the sensor and providing detection signals by means of at least two sensors (19, 20) of the sensor device (18) arranged at a distance from each other in longitudinal direction of the fish receiving space.

23. Method according to claim 22, characterised by determining the fish length on the basis of the detection signals by means of an evaluation device of the sensor device (18).

24. Method according to claim 23, characterised by checking, on the basis of the detection signals by means of the evaluation unit, whether when detecting the presence of the fish in the sensing region of the first sensor (19) arranged on the stunning means side the presence of the fish in the sensing region of the second sensor (20) arranged on the fish supply device side is detected as well, and, if the presence of the fish in both sensing regions of the first and second sensors (19, 20) is detected, by determining as fish size the respective larger stipulated fish size range, and by otherwise determining the respective smaller of the stipulated fish size ranges.

25. Method according to claim 23, characterised by detecting the presence of the fish on the respective sensing region of the sensor and providing detection signals by means of further sensors arranged at a distance from each other in longitudinal direction of the fish receiving space (10) and, by means of the evaluation device, determining the respective fish size range as the fish size on the basis of the detection signals, if the fish size falls into a fish size range corresponding to the distance between the first sensor and one of the further sensors.

26. Method according to one of the claims 19 to 25, characterised in that the fish are guided to slide into a floor element (21) of the fish receiving space (10) after loading the fish receiving space (10) under the effect of gravity, wherein the floor element (21) forms an inclined plane tilted towards the stunning means (13) in the direction of gravity.

27. Method according to claim 26, characterised by determining at least one time interval, which the fish needs to move from one sensing region of one of the sensors to at least one other sensing region of at least one of the other sensors, by means of a time difference sensing unit of the fish size sensing device on the basis of the detection signals of at least two of the sensors, and determining the fish length on the basis of a cinematic model describing the motion of the fish in the fish receiving space (10) by means of a fish processing unit of the fish size sensing device.

28. Method according to claim 27, characterised in that the cinematic model comprises at least one path/time equation, which at least approximately describes the motion of the fish depending on an angle of inclination of the inclined plane.

29. Method according to one of the claims 27 or 28, characterised in that the cinematic model comprises empirically determined motion data.

30. Method according to one of the claims 27 or 29, characterised in that the cinematic model is generated on the basis of a self-learning neuronal network.

31. Method according to one of the claims 19 to 30, characterised by initiating the fish size sensing device to firstly determine the fish size of the fish after loading the fish receiving space (10), and to subsequently set the position of the fish head relative to the stunning means in the stop position depending on the fish size by means of the controllably moveable position actuator in a self-actuating manner.

32. Method according to one of the claims 19 to 31, characterised by determining a head-to-overall-length relationship based on the fish size by means of the fish length sensing device and setting the position of the fish head in the stop position depending on the head-to-overall length relationship by means of the control unit.

33. Method according to one of the claims 19 to 32, characterised by determining by means of a prone/supine position sensing unit of the control unit whether the fish is aligned with its abdominal side upwards, and if the fish is aligned with its abdominal side upwards, separating out the fish from the fish receiving space (10).

34. Method according to claim 33, characterised by detecting sexual dimorphism-related changes in the fish by means of a kype recognition device of the control unit and adjusting the determined head-to-overall-length relationship based on an anatomic model of the fish according to the determined kype condition of the fish.

35. Method according to claim 34, characterised by an optical scanning of the abdominal side of the fish by means of an optical sensor of the kype recognition device and determining a brightness value or a brightness profile as well as a comparing the same with a stipulated brightness value or brightness profile in order to detect the kype condition.

36. Method according to one of the claims 19 to 35, characterised by determining fish size ranges by means of the fish size sensing device based on a stipulated distribution of the fish sizes and/or on a determined fish size distribution, and if the respective determined fish size lies within one of these fish size ranges, by setting the position of the fish head in the stop position depending on a position stipulation stipulated for the respective fish size range.
